(19) United States
(12) Patent Application Publication
(10) Pub. No.: US 2006/0188496 A1
(43) Pub. Date: Aug. 24, 2006

(54) Intranasal administration of active agents to the central nervous system

(76) Inventors: Hanne Bentz, Newark, CA (US); Beth Hill, Sunnyvale, CA (US); Catherine Lucas, Millbrae, CA (US); William H. Frey II, White Bear Lake, MN (US)

Correspondence Address: PERKINS COIE LLP, P.O. BOX 2168, MENLO PARK, CA, 94026, US

(21) Appl. No.: 11/342,058

(22) Filed: Jan. 27, 2006

Related U.S. Application Data
(60) Provisional application No. 60/655,809, filed on Feb. 23, 2005.

(51) Int. Cl. A61K 38/17; A61K 39/395

(52) U.S. Cl. 424/133.1; 514/12

(57) ABSTRACT

A method for delivering a polypeptide to the central nervous system of a mammal is provided. The method involves attaching the polypeptide to an antibody or an antibody fragment and administering the fusion polypeptide intranasally, for delivery to the central nervous system. Methods of treatment are also provided, where a therapeutically effective amount of the composition is delivered to the nasal cavity of a mammal.

### CROSS-REFERENCE TO RELATED APPLICATIONS

This application claims the benefit of U.S. Provisional Application No. 60/655,809, filed Feb. 23, 2005, incorporated herein by reference in its entirety.

### TECHNICAL FIELD

The subject matter described herein relates to methods of intranasal administration of active agents to the central nervous system of a mammal.

### BACKGROUND

Delivery of drugs to the central nervous system (CNS) remains a challenge, despite recent advances in drug delivery and knowledge of mechanisms of delivery of drugs to the brain. For example, CNS targets are poorly accessible from the peripheral circulation due to the blood-brain barrier (BBB), which provides an efficient barrier for the diffusion of most, especially polar, drugs into the brain from the circulating blood. Attempts to circumvent the problems associated with the BBB to deliver drugs to the CNS include: 1) design of lipophilic molecules, as lipid soluble drugs with a molecular weight of less than 600 Da readily diffuse through the barrier; 2) binding of drugs to transporter molecules which cross the BBB via a saturable transporter system, such as transferrin, insulin, IGF-1, and leptin; and 3) binding of drugs to polycationic molecules such as positively-charged proteins that preferentially bind to the negatively-charged endothelial surface (See, e.g., Illum, Eur. J. Pharm. Sci. 11:1-18 (2000) and references therein; W. M. Partridge. “Blood-brain barrier drug targeting: the future of brain drug development”, Mol Interv. 3(2):90-105 (2003); W. M. Partridge et al., “Drug and gene targeting to the Brain with molecular Trojan horses”, Nature Reviews-Drug Discovery 1:131-139 (2002)).

The intranasal route has been explored as a non-invasive method to circumvent the BBB for transport of drugs to the CNS. Although intranasal delivery to the CNS has been demonstrated for a number of small molecules and some peptides and smaller proteins, there is little evidence demonstrating the delivery of protein macromolecules to the CNS via intranasal pathways, presumably due to the larger size and varying physico-chemical properties unique to each macromolecule or class of macromolecules, that may hinder direct nose-to-brain delivery.

The primary physical barrier for intranasal delivery is the respiratory and olfactory epithelia of the nose. It has been shown that the permeability of the epithelial tight junctions in the body is variable and is typically limited to molecules with a hydrodynamic radius less than 3.6 A; permeability is thought to be negligible for globular molecules with a radius larger than 15 A (B. R. Stevenson et al., Mol. Cell. Biochem. 83, 129-145(1988)). Therefore, the size of the molecule to be administered is considered an important factor in achieving intranasal transport of a macromolecule to the central nervous system. Fluorescein-labeled dextran, a linear molecule having a dextran molecular weight of 20 kD can be delivered to cerebrospinal fluid from the rat nasal cavity, however 40 kDa dextran cannot (Sakane et al, J. Pharm. Pharmacol. 47, 379-381 (1995)). It has also been reported that an infectious organism, such as a virus, can enter the brain through the olfactory region of the nose (S. Perlman et al., Adv. Exp. Med. Biol., 380:73-78 (1995)). In published delivery studies to date, intranasal delivery efficiency to the CNS has been very low and the delivery of large globular macromolecules, such as antibodies and their fragments, has not been demonstrated. Yet, because antibodies, antibody fragments, and antibody fusion molecules are potentially useful therapies for treating disorders having CNS a target, e.g., Alzheimer's disease, Parkinson's disease, multiple sclerosis, stroke, epilepsy, and metabolic and endocrine disorders, it is desirable to provide a method for delivering these large macromolecules to the CNS non-invasively.

The foregoing examples of the related art and limitations related therewith are intended to be illustrative and not exclusive. Other limitations of the related art will become apparent to those of skill in the art upon a reading of the specification and a study of the drawings.

### BRIEF SUMMARY

It has been discovered that globular protein molecules, such as an antibody fragment linked to a therapeutic peptide or protein, can be delivered directly to the central nervous system of a mammal, thereby bypassing the blood-brain barrier. Accordingly, methods of delivering a therapeutic composition to the central nervous system of a mammal are provided. The methods are advantageous in treating a wide variety of diseases or conditions. Methods of treatment are therefore also provided.

In a first aspect, a method of delivering a therapeutic composition to the central nervous system of a mammal is provided. The method includes intranasally administering a therapeutic composition to the mammal, wherein the therapeutic composition is comprised of a therapeutically effective amount of an antibody fragment and a polypeptide. In one embodiment, the antibody fragment is linked to the polypeptide.

In one embodiment, intranasal administration achieves uptake of the therapeutic composition via absorption across nasal epithelial tissue, for example the olfactory epithelium, for delivery of the therapeutic composition via the olfactory and/or trigeminal neural pathways.

In another aspect, a method for targeting a polypeptide to the CNS by attaching the polypeptide to an antibody or an antibody fragment to form a fusion polypeptide, and administering the fusion polypeptide intranasally is provided. In one embodiment, the polypeptide is biologically active and provides a therapeutic benefit. In another embodiment, the antibody or antibody fragment biologically active and provides a therapeutic benefit, in addition to having binding affinity for an endogenous target, such as a cell or tissue.

In a third aspect, methods of treatment are provided, where intranasal administration of the therapeutic composition is provided for treatment of a condition that responds to or requires delivery of the therapeutic compound to the CNS.

These and other aspects and embodiments will be apparent from the description, drawings, and sequences herein.

### BRIEF DESCRIPTION OF THE FIGURES

FIG. 1 is a graph showing the distribution of ¹²⁵I-α-melanocyte stimulating hormone (¹²⁵I-α-MSH) mimetibody in rats 25 minutes (open bars) and 5 hours (dotted bars) after intranasal administration of ¹²⁵ I-α-MSH mimetibody, as more fully described in Example 1.

FIG. 2 is a graph showing the blood concentration of ¹²⁵I-α-MSH mimetibody, in nmol, after intranasal (diamonds) or intravenous (squares) administration of ¹²⁵I-α-MSH mimetibody to rats, as a function of time post delivery, in minutes, as more fully described in Example 1.

FIG. 3 is a graph comparing the distribution of ¹²⁵I-α-MSH mimetibody in the central nervous system and peripheral tissues of rats after either intranasal (open bars) or intravenous (dotted bars) administration of ¹²⁵I-α-MSH mimetibody, as more fully described in Example 1.

FIGS. 4A-4D show computer-generated autoradiographs of coronal sections of rat brains 25 minutes after administration of ¹²⁵I-α-MSH mimetibody either intranasally (FIGS. 4A, 4C) or intravenously (FIGS. 4B, 4D), as more fully described in Example 1.

FIG. 5 is a graph showing the reduction of cumulative food intake in rats, in grams, 24 hours after intranasal treatment with α-MSH mimetibody at varying doses, in nmol.

FIG. 6 is a graph showing the percentage reduction in cumulative food intake in rats, as a function of time, in hours, after intranasal treatment with α-MSH mimetibody at a dose of 2.5 nmol (diamonds), 6.25 nmol (squares), 25 nmol (triangles), or 50 nmol (circles), FIG. 7 is a bar graph showing the cumulative food intake in rats, in grams, at the indicated times post treatment with α-MSH mimetibody (open bars) or saline (dotted bars) administered intranasally.

### DETAILED DESCRIPTION

For the purposes of promoting an understanding of the subject matter herein, reference will now be made to preferred embodiments and specific language will be used to describe the same. It will nevertheless be understood that no limitation of the scope of the invention is thereby intended, such alterations and further modifications of the subject matter, and such further applications of the principles as illustrated herein, being contemplated as would normally occur to one skilled in the art to which the subject matter relates.

Methods of delivering therapeutic compositions to the central nervous system, including the brain and spinal cord, of a mammal by a non-systemic route, e.g., by a route other than one which delivers or otherwise affects the body as a whole are provided. The delivery method therefore allows for localized and targeted delivery of the therapeutic compositions to the brain via the nasal passage. Consequently, the method relates to delivery of the compositions by a route other than intravenous, intramuscular, transdermal, intraperitoneal, or similar route which delivers the composition through, for example, the blood circulatory system. It has been discovered that antibody fragments conjugated or otherwise linked to a therapeutic polypeptide may be delivered to the central nervous system, including the brain and spinal cord, of a mammal by administration of the fusion molecule intranasally.

As used herein, the term “polypeptide” intends a polymer of amino acids and does not refer to a specific length of a polymer of amino acids. Thus, for example, the terms peptide, oligopeptide, protein, and enzyme are included within the definition of polypeptide. This term also includes post-expression modifications of the polypeptide, for example, glycosylations, acetylations, phosphorylations, and the like. In some instances, the terms protein, peptide, and polypeptide are used interchangeably.

The compositions are applied intranasally such that the compositions will be transported to the brain directly, such as by a non-systemic route. Accordingly, methods of delivering therapeutic compositions to the central nervous system of a mammal are provided herein. Methods of treating a disorder responsive to treatment by application of a therapeutic composition to the central nervous system of a mammal are also provided and described below.

A. Composition Components

The therapeutic composition for intranasal delivery is a fusion polypeptide comprised of polypeptide and an antibody or antibody fragment. In one embodiment, the polypeptide is biologically active and preferably causes or otherwise brings about a particular biological effect, such as a therapeutic effect. Various example of polypeptides are given below. The polypeptide is linked to an antibody or antibody fragment directed against an endogenous target. The antibody or antibody fragment, in addition to having binding affinity for a cellular target, may be biologically active to cause a therapeutic effect. Together the polypeptide and the attached antibody or antibody fragment comprise a therapeutic compound or therapeutic fusion polypeptide, that can be formulated as desired for intranasal delivery. As will be illustrated below, the increased size and/or hydrophilicity of the fusion polypeptide, relative to the individual components, reduces the blood bioavailability of the polypeptide while allowing delivery to the central nervous system, thus improving drug targeting while reducing systemic exposure and associated side effects.

i. Antibody or Antibody Fragment

The antibody or antibody fragment in the therapeutic fusion compound may be selected to serve as a targeting agent, to provide a biologically desired effect, or both. The antibody or antibody fragment may be a polyclonal or a monoclonal antibody, and exemplary antibodies and fragments, sources of and preparation of the same, are now described.

Polyclonal antibodies may be obtained by injecting a desired antigen into a subject, typically an animal such as a mouse, as well established in the art. The antigen is selected based on the disorder to be treated. For example, in treating Alzheimer's disease, the antigen may be β-amyloid protein or peptides thereof. In treating cancer, the antigen may be a tumor-associated antigen, such as various peptides known to the art, including, for example, interleukin-13 receptor-α (for malignant astrocytoma/glioblastoma multiforme as discussed in Joshi, B. H. et al., Cancer Res. 60:1168-1172 (2000)), BF7/GE2 (microsomal epoxide hyrdrolase; mEH) (for treatment of tumors with abnormal mEH expression as discussed in Kessler, R. et al., Cancer Res. 60:1403-1409 (2000)), tyrosinase-related protein-2 (TRP-2) (for treatment of glioblastoma multiforme), MAGE-1, 3 or 6 (for medulloblastomas) and MAGE-2 (for glioblastoma multiforme) (both as discussed in Scarcella, D. L., et al., Clin. Cancer Res., 5:331-341 (1999)), and survivin (for medulloblastomas as described in Bodey, B. B., In Vivo, 18(6)713-718 (2004)). For treatment of neurotrauma to suppress inflammation such as in spinal cord injury and acute brain injury, the antigen may be-TNF-alpha and various interleukins, including interleukin-1□. The antigen, along with an adjuvant such as Freund's complete adjuvant, may be injected into the subject multiple times subcutaneously or intraperitoneally.

Another method to increase the immunogenicity of the antigen is to conjugate or otherwise link the antigen to a protein that is immunogenic in the particular species which will produce the antibodies. For example, the antigen may be conjugated to polytuftsin (TKPR40), a synthetic polymer of the natural immunomodulator tuftsin, which has been shown to increase the immunogenicity of synthetic peptides in mice (Gokulan K. et al., DNA Cell Biol. 18(8):623-630 (1999)). The method of conjugation may involve use of a bifunctional or derivatizing agent, such as maleimidobenzoyl sulfosuccinimide ester for conjugation through cysteine residues, N-hydroxysuccinimide for conjugation through lysine residues, glutaradehyde or succinic anhydride.

After a sufficient period of time after the initial injection, such as, for example, about one month, the animals may be boosted with a fraction of the original amount of peptide antigen, such as 1/10 the amount, and may then be bled about 7 to 14 days later and the antibodies may be isolated from the blood of the animals by standard methods known to the art, including affinity chromatography using, for example, protein A or protein G sepharose; ion-exchange chromatography, hydroxylapatite chromatography or gel electrophoresis. Antibody purification procedures may be found, for example, in Harlow, D. and Lane E., Using Antibodies: A Laboratory Manual, Cold Springs Harbor Laboratory Press, Woodbury, N.Y. (1998); and Subramanian, G., Antibodies: Production and Purification, Kluwer Academic/Plenum Publishers, New York, N.Y. (2004).

Non-human antibodies may be humanized by a variety of methods. For example, hypervariable region sequences in the non-human antibodies may be substituted for the corresponding sequences of a human antibody as described, for example, in Jones et al., Nature, 321:522-525 (1986); Reichmann et al., Nature, 332:323-327 (1988) and Verhoeyen et al., Science, 239:1534-1536 (1988). As the antibody is intended for human therapy, it is preferable to select a human variable domain for guidance in making a humanized antibody, in order to reduce the antigenicity of the antibody. In order to accomplish this, the sequence of the variable domain of the non-human antibody may be screened against a library of known human variable domain sequences. The human variable domain sequence which is the closest match to that of the animal is identified and the human framework region within it is utilized in the human antibody as described, for example, in Sims et al., J. Immunol., 151:2296-2308 (1993) and Chothia et al., J. Mol. Biol., 196:901-917 (1987).

The antibody may be a full length antibody or a fragment. The full length antibody or fragment may be modified to allow for improved stability of the antibody or fragment and to modulate effector function, such as binding to an Fc receptor. This may be achieved, for example, by utilizing human or murine isotypes, or variants of such molecules such as IgG4 with Ala/Ala mutations, to lose effector function and yet still maintain IgG structure. The antibody fragment may be a monomer or a dimer, and includes Fab, Fab′, F(ab′)2, Fc, or an Fv fragment. These fragments may be produced, for example, by proteolytic degradation of the intact antibody. For example, digestion of intact antibodies with papain results in two Fab fragments. Treatment of intact antibodies with pepsin provides a F(ab′)2 fragment. The F(ab′)2 fragment is a dimer of Fab, which is a light chain joined to VH-CH1 by a disulfide bond. The F(ab)′2 may be reduced under mild conditions to break the disulfide linkage in the hinge region, thereby converting the (Fab′)2 dimer into a Fab′ monomer. The Fab′ monomer is essentially a Fab fragment with part of the hinge region (see Fundamental Immunology, W. E. Paul, ed., Raven Press, N.Y. (1993), for a more detailed description of other antibody fragments).

Many fragments, including those that have the Fc portion, can also be produced by recombinant DNA technology methods known to the art.

A wide variety of antibodies may be used to obtain the antibody fragments utilized in the compositions for intranasal delivery to the central nervous system described herein. Exemplary antibodies include IgG, IgM, IgA, IgD, and IgE. Subclasses of these antibodies may also be used to obtain the antibody fragments. Exemplary subclasses include IgG1, IgG2, IgG3, IgG4, IgA1 and IgA2. The antibody fragments may be obtained by proteolytic degradation of the antibodies which may be produced as previously discussed herein. In one embodiment, the antibody fragment is utilized to increase the half-life of the polypeptide, and antibodies may be isolated from a subject without immunization and may be isolated by antibody isolation procedures previously described herein. Antibody fragments may alternatively be produced by recombinant DNA methods as previously described herein, in order to produce chimeric or fusion polypeptides. For example, a fusion molecule may be produced utilizing a plasmid encoding the respective proteins to generate the mimetibody, which includes the antibody fragment and the therapeutic polypeptide.

Antibodies, antibody fragments or antibody fragments linked to polypeptides, or biologically active portions thereof, may be purified by affinity purification including use of a Protein A column and size exclusion chromatography utilizing, for example, Superose columns. Purification methods are well known in the art.

Specific monoclonal antibodies may be prepared by the technique of Kohler and Milstein, Eur. J. Immunol., 6:511-519 (1976) and improvements and modifications thereof. Briefly, such methods include preparation of immortal cell lines capable of producing desired antibodies. The immortal cell lines may be produced by injecting the antigen of choice into an animal, such as a mouse, harvesting B cells from the animal's spleen and fusing the cells with myeloma cells to form a hybridoma. Colonies may be selected and tested by routine procedures in the art for their ability to secrete high affinity antibody to the desired epitope. After the selection procedures, the monoclonal antibodies may be separated from the culture medium or serum by antibody purification procedures known to the art, including those procedures previously described herein.

Alternatively, antibodies may be recombinantly produced from expression libraries by various methods known in the art. For example, cDNA may be produced from ribonucleic acid (RNA) that has been isolated from lymphocytes, preferably from B lymphocytes and preferably from an animal injected with a desired antigen. The cDNA, such as that which encodes various immunoglobulin genes, may be amplified by the polymerase chain reaction (PCR) and cloned into an appropriate vector, such as a phage display vector. Such a vector may be added to a bacterial suspension, preferably one that includes E. coli, and bacteriophages or phage particles may be produced that display the corresponding antibody fragment linked to the surface of the phage particle. A sublibrary may be constructed by screening for phage particles that include the desired antibody by methods known to the art, including, for example, affinity purification techniques, such as panning. The sublibrary may then be utilized to isolate the antibodies from a desired cell type, such as bacterial cells, yeast cells or mammalian cells. Methods for producing recombinant antibodies as described herein, and modifications thereof, may be found, for example, in Griffiths, W. G. et al., Ann. Rev. Immunol., 12:433-455 (1994); Marks, J. D. et al., J. Mol. Biol., 222:581-597 (1991); Winter, G. and Milstein, C., Nature, 349:293-299 (1991); and Hoogenboom, H. R. and Winter, G., J. Mol. Biol., 227(2):381-388 (1992).

Human antibodies may also be produced in transgenic animals. For example, homozygous deletion of the antibody heavy chain joining region (J_{H}) gene in chimeric and germ-line mutant mice results in complete inhibition of endogenous antibody production such that transfer of a human germ-line immunoglobulin gene array into such mutant mice results in production of human antibodies when immunized with antigen. See, e.g., Jakobovits et al., Proc. Natl. Acad. Sci. USA, 90:2551-2551 (1993); Jakobovits et al., Nature, 362:255-258 (1993); U.S. Pat. Nos. 5,545,806; 5,569,825; 5,591,669; 5,545,807 and PCT publication WO 97/17852.

ii. Polypeptide

As noted above, the antibody or antibody fragment is linked to a polypeptide. Preferably, the polypeptide is one that may bind to a region of the central nervous system. The polypeptide is further preferably one that has a beneficial effect on the central nervous system, and includes one that has a beneficial effect on functions regulated by the central nervous system of a mammal, such as for therapeutic purposes. The polypeptide may exert its effects by binding to, for example, cellular receptors in various regions of the brain. As one example, in order for α-melanocyte stimulating hormone (α-MSH) to exert its effect in body weight reduction, it binds to the melanocortin 4 receptor (MCR-4) on neurons in the hypothalamus. As a further example, in order for erythropoietin (EPO), active EPO fragments or EPO analogs to improve neurologic function after stroke or acute brain injury, it has to bind to neuronal receptors, e.g., on hippocampal cells, astrocytes, or similar cells.

A wide variety of proteins or peptides may be utilized. The polypeptides may have a molecular weight of about 200 Daltons to about 200,000 Daltons, but are typically about 300 Daltons to about 100,000 Daltons.

In one embodiment, the polypeptide and antibody or antibody fragment, after attachment, have a combined molecular weight of greater than about 25 kDa, more preferably of greater than about 30 kDa, still more preferably of greater than about 40 kDa.

In another embodiment, the polypeptide has a molecular weight of less than about 25 kDa and is hydrophobic.

A wide variety of therapeutic proteins, or biologically active portions thereof, may be linked or otherwise attached to the antibody fragments that may be utilized in the methods described herein. The proteins are preferably in the form of peptides. The specific therapeutic peptide selected will depend on the disease or condition (collectively referred to as “disorder”) to be treated. For neurodegenerative disorders, such as, for example, Alzheimer's disease, Parkinson's disease and Huntington's disease, or other disease involving loss of locomotion or cognitive function such as memory, neuroprotective or neurotrophic agents are preferred. The neuroprotective or neurotrophic agent may be one that promotes neuronal survival, stimulates neurogenesis and/or synaptogenesis, rescues hippocampal neurons from beta-amyloid-induced neurotoxicity and/or reduces tau phosphorylation. Examples of agents suitable for treating such neurodegenerative disorders, and neurological disorders, include leutenizing hormone releasing (LHRH) and agonists of LHRH, such as deslorelin; neurotrophic factors, such as those from the neurotrophin family, including nerve growth factor (NGF), brain-derived neurotrophic factor (BDNF), neurotrophin-3 and neurotrophin-4/5; the fibroblast growth factor family (FGFs), including acidic fibroblast growth factor and basic fibroblast growth factor; the neurokine family, including ciliary neurotrophic factor, leukemia inhibitory factor, and cardiotrophin-1; the transforming growth factor-β family, including transforming growth factor-β-1-3 (TGF-betas), bone morphogenetic proteins (BMPs), growth/differentiation factors such as growth differentiation factors 5 to 15, glial cell line-derived neurotrophic factor (GDNF), neurturin, artemin, activins and persephin; the epidermal growth factor family, including epidermal growth factor, transforming growth factor-α and neuregulins; the insulin-like growth factor family, including insulin-like growth factor-1 (IGF-1) and insulin-like growth factor-2 (IGF-2); the pituitary adenylate cyclase-activating polypeptide (PACAP)/glucagons superfamily, including PACAP-27, PACAP-38, glucagons, glucagons-like peptides such as GLP-1 and GLP-2, growth hormone releasing factor, vasoactive intestinal peptide (VIP), peptide, histidine methionine, secreting and glucose-dependent insulinotropic polypeptide; and other neurotrophic factors, including activity-dependent neurotrophic factor and platelet-derived growth factors (PDGFs). Such agents are also suitable for treating acute brain injury, chronic brain injury (neurogenesis) and neuropsychologic disorders, such as depression.

In the case of stroke treatment, the therapeutic agent may be one that protects cortical neurons from nitric oxide-mediated neurotoxicity, promotes neuronal survival, stimulates neurogenesis and/or synaptogenesis and/or rescues neurons from glucose deprivation. Examples of such agents include the neurotrophic factors previously described herein, active fragments thereof, as well as erythropoietin (EPO), analogs of EPO, such as carbamylated EPO, and active fragments of EPO. Examples of EPO analogs that may be used include those known to the skilled artisan and described, for example, in U.S. Pat. Nos. 5,955,422 and 5,856,298. Peptide growth factor mimetics of, and antagonists to, for example, EPO, granulocyte colony-stimulating factor (GCSF), and thrombopoietin useful in the invention can be screened for as reviewed by K. Kaushansky, Ann. NY Acad. Sci., 938:131-138 (2001) and as described for EPO mimetic peptide ligands by Wrighton et al., Science, 273(5274):458-450 (1996). The mimetics, agonists and antagonists to the peptide growth factors, or other peptides or proteins described herein, may be shorter in length than the peptide growth factor or other polypeptide that the mimetic, agonist or antagonist is based on.

Therapeutic polypeptides for treatment of eating disorders, such as for prevention of weight loss (anorexia) and weight gain (obesity), include melanocortin receptor (MCR) agonists and antagonists. Suitable MCR agonists include α-melanocyte stimulating hormone (α-MSH) as well as beta and gamma—MSH, and derivatives thereof, including amino acids 1 to 13 of human α-MSH (SEQ ID NO:1 SYSMEHFRWGKPV) and specifically receptor binding amino acid sequence 4-10, as in adrenocorticotropic hormone (MSH/ACTH₄₋₁₀) melanocortin receptor-3 (MCR3) or melanocortin receptor 4 (MCR4) agonists, such as melanotan II (MTII), a potent non-selective MCR agonist, MRLOB-0001 and active fragments of the peptides and/or proteins. Other peptides for obesity treatment include hormone peptide YY (PYY), especially amino acids 3 to 36 of the peptide, leptin and ghrelin, ciliary neurotrophic factor or analoqs thereof, glucagon-like peptide-1 (GLP-1), insulin mimetics and/or sensitizers, leptin, leptin analogs and/or sensitizers and dopaminergic, noradrenergic and serotinergic agents.

Corresponding MCR antagonists regulating body weight homeostasis include endocannabinoid receptor antagonists, fatty acid synthesis receptor inhibitors, ghrelin antagonists, melanin-concentrating hormone receptor antagonists, PYY receptor antagonists and tyrosine phosphatase-1B inhibitors (J. Korner et al., J. Clin. Invest., 111:565-570 (2003)). MCR antagonists, such as Agouti signaling protein (ASIP) and Agouti-related protein (AGRP), which are endogenous MCR3 and MCR4 antagonists, and their peptoid variants and mimetics may be used to control body weight homeostasis and to treat eating disorders such as anorexia (Y K Yang et al., Neuropeptides, 37(6):338-344 (2003); D A Thompson et al, Bioorg Med Chem Lett., 13:1409-1413 (2003); and C. Chen et al, J. Med. Chem., 47(27):6821 -30 (2004)).

The previously mentioned peptide hormones and analogs thereof that bind to melanocortin receptors (MCRs) may also be useful to control inflammation and improve male and female sexual dysfunction (A. Catania et al., Pharmacol Rev, 56(1): 1-29 (2004)).

The therapeutic protein for treatment of endocrine disorders, such as diabetes mellitus includes, for example, glucagon-like peptide 1 (GLP-1); peptides from the GLP-1 family, including pituitary adenylate cyclase-activating polypeptide (PACAP), vasoactive intestinal peptide (VIP), exendin-3 and exendin4;and insulin-like growth factor (IGF-1), IGF binding protein 3 (IGFBP3) and insulin, and active fragments thereof.

The therapeutic polypeptide for treatment of sleep disorders, such as insomnia, includes growth hormone releasing factor, vasopressin, and derivatives of vasopressin, including desmopressin, glypressin, ornipressin and ternipressin; Included are peptide variants and mimetic peptide ligands that bind to the same receptor targets resulting in either the same/similar or the opposite biological response. The therapeutic protein for treatment of autoimmune disorders, such as multiple sclerosis, includes interferons, including β-interferon, and transforming growth factor β's.

The therapeutic polypeptide for treatment of psychiatric disorders, such as schizophrenia, includes neuregulin-1, EPO, analogs of EPO, such as carbamylated EPO, and active fragments of EPO and EPO mimetics as previously described herein. Various neurotrophic factors and regulatory peptide hormones, such as brain-derived neurotrophic factor (BDGF) and insulin, may be used to treat depression, and psychoendocrinologic and metabolic disorders.

The therapeutic polypeptide for treatment of lysosomal storage disorders of the brain includes, for example, lysosomal enzymes.

The therapeutic polypeptide for treatment of eating disorders such as anorexia includes, for example, melanocortin receptor (MCR) antagonists such as Agouti signaling protein (ASIP) and Agouti related protein (AGRP).

The therapeutic polypeptides may be human polypeptides, although the polypeptides may be from other species or may be synthetically or recombinantly produced. The original amino acid sequence may also be modified or reengineered such as for improved potency or improved specificity (e.g. eliminate binding to multiple receptors) and stability.

Therapeutic polypeptides utilized herein may also be mimetics, such as molecules that bind to the same receptor but have amino acid sequences that are non-homologous to endogenous human peptides. For example, the agonist and antagonists, including agonists and antagonists of melanocortin receptor, growth hormone releasing factor receptor, vasopressin receptor, hormone peptide YY receptor, a neuropeptide Y receptor, or erythropoietin receptor, may include natural amino acids, such as the L-amino acids or non-natural amino acids, such as D-amino acids. The amino acids in the polypeptide may be linked by peptide bonds or, in modified peptides, including peptidomimetics, by non-peptide bonds (J. Zhang et al., Org. Lett., 5(17): 3115-8 (2003)).

Polypeptide mimetics, and receptor agonists and antagonists can be selected and produced utilizing high throughput screening known to the art for specific biological function and receptor binding. The availability of such methods allows rapid screening of millions of randomly produced organic compounds and peptides to identify lead compounds for further development. Strategies used to screen libraries of small molecules and peptides and the success in finding mimetics and antagonists, e.g., for/to EPO, GCSF and thrombopoietin, are reviewed by K. Kaushansky, Ann. NY Acad. Sci., 938:131-138 (2001).

A wide variety of modifications to the amide bonds which link amino acids may be made to the agonists and antagonists described herein, and such modifications are well known in the art. For example, such modifications are discussed in general reviews, including in Freidinger, R. M. “Design and Synthesis of Novel Bioactive Peptides and Peptidomimetics” J. Med. Chem., 46:5553 (2003), and Ripka, A. S., Rich, D. H. “Peptidomimetic Design” Curr. Opin. Chem. Biol., 2:441 (1998). Many of the modifications are designed to increase the potency of the peptide by restricting conformational flexibility.

For example, the agonists and antagonists may be modified by including additional alkyl groups on the nitrogen or alpha-carbon of the amide bond, such as the peptoid strategy of Zuckerman et al, and the alpha modifications of, for example Goodman, M. et. al. (Pure Appl. Chem., 68:1303 (1996)). The amide nitrogen and alpha carbon may be linked together to provide additional constraint (Scott et al, Org. Letts., 6:1629-1632 (2004)).

iii. Linkages

The polypeptide is linked to the antibody or antibody fragment to form the therapeutic compound for delivery. The antibody or antibody fragment, in one embodiment, increases the stability of the polypeptide, thereby increasing its half life in vivo, including in the nasal cavity and the central nervous system of a mammal. The combined polypeptide-antibody fragment compound is also referred to herein as a “mimetibody”. In this section, approaches for linking the two moieties is described.

The antibody fragment and polypeptide may be linked to each other by methods known to the art, and typically through covalent bonding. The linking or conjugation method may include use of amino acid linkers, including use of glycine and serine. The fragment and polypeptide may be conjugated or otherwise linked by cross-linking or other linking procedures know to the art and discussed, for example, in Wong, S. S., Chemistry of Protein Conjugation and Cross-Linking, CRC Press, Boca Raton, Fla. (1991). For example, the polypeptides may be conjugated utilizing homo-bifunctional and/or hetero-bifunctional or multifunctional cross-linkers known to the art. Examples of cross-linking agents include carbodiimides, such as EDC (1-Ethyl-3-[3-dimethylaminopropyl]carbodiimide hydrochloride); imidoesters, N-hyroxysuccinimide-esters, maleimides, pyridyl disulfides, hydrazides and aryl azides. Several points of attachment between the active agent polypeptide and the antibody fragment are envisioned, including linkage of the N-terminus of the peptide to the C-terminus of the antibody fragment. The polypeptide may, alternatively, be attached at its C-terminus to the N-terminus of the antibody fragment. Conjugation may further be via cysteine or other amino acid residues or via a carbohydrate functional moiety of the antibody.

iv. Formulation of the Therapeutic Polypeptide-Antibody Compound

The active agent polypeptide in the therapeutic composition may be mixed with a pharmaceutically-acceptable carrier or other vehicle. The carrier may be a liquid suitable, for example, for administration as nose drops or as a nose spray, and includes water, saline or other aqueous or organic and preferably sterile solution. The carrier may be a solid, such as a powder, gel or ointment and may include inorganic fillers such as kaolin, bentonite, zinc oxide, and titanium oxide; viscosity modifiers, antioxidants, pH adjusting agents, lyoprotectants and other stability enhancing excipients, including sucrose, antioxidants, chelating agents; humectants such as glycerol, and propylene glycol; and other additives which may be incorporated as necessary and/or desired.

Where the therapeutic compound is administered as a gel or ointment, the carrier may include suitable solid, such as a pharmaceutically acceptable base material known for use in such carriers, including, for example, natural or synthetic polymers such as hyaluronic acid, sodium alginate, gelatin, corn starch, gum tragacanth, methylcellulose, hydroxyethylcellulose, carboxymethylcellulose, xanthan gum, dextrin, carboxymethylstarch, polyvinyl alcohol, sodium polyacrylate, methoxyethylene maleic anhydride copolymer, polyvinylether, polyvinylpyrrolidone; fats and oils such as beeswax, olive oil, cacao butter, sesame oil, soybean oil, camellia oil, peanut oil, beef fat, lard, and lanolin; white petrolatum; paraffins; hydrocabon gel ointments; fatty acids such as stearic acid; alcohols such as cetyl alcohol and stearyl alcohol; polyethylene glycol; and water.

Where the therapeutic compound is administered as a powder, the carrier may be a suitable solid such as oxyethylene maleic anhydride copolymer, polyvinylether, polyvinylpyrrolidone polyvinyl alcohol; polyacrylates, including sodium, potassium or ammonium polyacrylate; polylactic acid, polyglycolic acid, polyvinyl alcohol, polyvinyl acetate, carboxyvinyl polymer, polyvinylpyrrolidone, polyethylene glycol; celluloses, including cellulose, microcrystalline cellulose, and.alpha.-cellulose; cellulose derivatives, including methyl cellulose, ethyl cellulose, hydroxyethyl cellulose, hydroxypropyl cellulose, hydroxypropyl methyl cellulose, sodium carboxymethyl cellulose and ethylhydroxy ethyl cellulose; dextrins, including alpha.-, beta.- or .gamma.-cyclodextrin, dimethyl-.beta.-cyclodextrin; starches, including hydroxyethyl starch, hydroxypropyl starch, carboxymethyl starch; polysaccharides, including dextran, dextrin and alginic acid; hyaluronic acid; pectic acid; carbohydrates, such as mannitol, glucose, lactose, fructose, sucrose, and amylose; proteins, including casein, gelatin, chitin and chitosan; gums, such as gum arabic, xanthan gum, tragacanth gum and glucomannan; phospholipids and combinations thereof.

The particle size of the powder may be determined by standard methods in the art, including screening or sieving through appropriately sized mesh. If the particle size is too large, the size can be adjusted by standard methods, including chopping, cutting, crushing, grinding, milling, and micronization. The particle size of the powders typically range from about 0.05 μm to about 100 μm. The particles are preferably no larger than about 400 μm.

The compositions may further include agents which improve the mucoadhesivity, nasal tolerance, or the flow properties of the composition, mucoadhesives, absorption enhancers, odorants, humectants, and preservatives. Suitable agents which increase the flow properties of the composition when in an aqueous carrier include, for example, sodium carboxymethyl cellulose, hyaluronic acid, gelatin, algin, carageenans, carbomers, galactomannans, polyethylene glycols, polyvinyl alcohol, polyvinylpyrrolidone, sodium carboxymethyl dextran and xantham gum. Suitable absorption enhancers include bile salts, phospholipids, sodium glycyrrhetinate, sodium caprate, ammonium tartrate, gamma.aminolevulinic acid, oxalic acid, malonic acid, succinc acid, maleic acid and oxaloacetic acid. Suitable humectants for aqueous compositions include, for example, glycerin, polysaccharides and polyethylene glycols. Suitable mucoadhesives include, for example, polyvinyl pyrrolidone polymer.

B. Nasal Delivery

The therapeutic composition, comprised of an antibody or antibody fragment linked to a polypeptide, may be administered by a wide variety of methods, and some exemplary methods are provided below. Absorption of the fusion polypeptide once introduced into the nasal cavity may occur via absorption across the olfactory epithelium, which is found in the upper third of the nasal cavity. Absorption may also occur across the nasal respiratory epithelium, which is innervated with trigeminal nerves, in the lower two-thirds of the nasal cavity. The trigeminal nerves also innervate the conjunctive, oral mucosa, and certain areas of the dermis of the face and head, and absorption after intranasal administration of the fusion polypeptide from these regions may also occur.

One exemplary formulation for intranasal delivery of the fusion polypeptide is a liquid preparation, preferably an aqueous based preparation, suitable for application as drops into the nasal cavity. For example, nasal drops can be instilled in the nasal cavity by tilting the head back sufficiently and apply the drops into the nares. The drops may also be snorted up the nose.

Alternatively, a liquid preparation may be placed into an appropriate device so that it may be aerosolized for inhalation through the nasal cavity. For example, the therapeutic agent may be placed into a plastic bottle atomizer. In one embodiment, the atomizer is advantageously configured to allow a substantial amount of the spray to be directed to the upper one-third region or portion of the nasal cavity. Alternatively, the spray is administered from the atomizer in such a way as to allow a substantial amount of the spray to be directed to the upper one-third region or portion of the nasal cavity. By “substantial amount of the spray” it is meant herein that at least about 50%, further at least about 70%, but preferably at least about 80% or more of the spray is directed to the upper one-third portion of the nasal cavity.

Additionally, the liquid preparation may be aerosolized and applied via an inhaler, such as a metered-dose inhaler. One example of a preferred device is that disclosed in U.S. Pat. No. 6,715,485 to Djupesland, and which involves a bi-directional delivery concept. In using the device, the end of the device having a sealing nozzle is inserted into one nostril and the patient or subject blows into the mouthpiece. During exhalation, the soft palate closes due to positive pressure thereby separating the nasal and oral cavities. The combination of closed soft palate and sealed nozzle creates an airflow in which drug particles are released entering one nostril, turning 180 degrees through the communication pathway and exiting through the other nostril, thus achieving bi-directional flow.

The fusion polypeptide can also be delivered in the form of a dry powder, as in known in the art. An example of a suitable device is the dry powder nasal delivery device marketed under the name DirectHaler™ nasal, and which is disclosed in PCT publication No. 96/222802. This device also enables closing of the passage between the nasal and oral cavity during dose delivery. Another device for delivery of a dry preparation is the device sold under the trade designation OptiNose™.

C. Methods of Treatment

In yet another aspect, methods of treatment are provided. The treatment methods may advantageously be utilized to treat a disorder in a mammal that is amenable to treatment by administration of a therapeutic agent to the central nervous system, such as the brain and/or spinal cord. That is, the disorder is one where the symptoms decrease or are otherwise eliminated, the rate of progression of the disorder decreases, and/or the disorder is eliminated by an agent that acts on the central nervous system.

In one embodiment, a method includes administering to the nasal cavity of a mammal, such as to cells and/or tissue in a region or portion of the nasal cavity of a mammal occupied by the superior turbinates, a therapeutically effective amount of an antibody fragment linked or otherwise conjugated to a polypeptide.

The method may be used to treat a wide variety of disorders. Suitable disorders include, for example, neurological and neurodegenerative disorders such as Alzheimer's disease, Parkinson's disease, and Huntington's disease; as well as other disorders known to the art that cause a loss of memory, such as multi-infarct dementia, Creutzfeldt-Jakob disease, Lewy body disease, normal pressure hydrocephalus and HIV dementia; or a loss of locomotion, such as stroke, amyotropic lateral sclerosis, myasthenia gravis and Duchenne dystrophy; endocrine, metabolic or energy balance disorders, such as obesity, diabetes and sleeping disorders, including insomnia; autoimmune disorders, such as multiple sclerosis; anorexia and treatment of acute injury from stroke or spinal cord injuries.

In one embodiment, a method of delivering a therapeutic composition to the central nervous system of a mammal includes administering the composition to the mammal intranasally, preferably to olfactory and/or trigeminal nerve endings, cells and nasal epithelium in a region of the nasal cavity located in the superior turbinates. This region or area is typically located in, but is not limited to, the upper one-third portion of the nasal cavity.

Although not being limited to any theory by which the method achieves its advantageous results, the agents that are applied intranasally according to the methods described herein may reach the brain directly by an extracellular or intracellular pathway. See, e.g., Thorne, R. G. et al., Neuroscience, 127:481-496 (2004). Intracellular pathways include transport through olfactory sensory neurons. This may involve, for example, absorptive or receptor-mediated endocytosis into olfactory sensory neurons and subsequent transport to olfactory bulb glomeruli. As another example, such transport may involve intraneuronal transport within the trigeminal nerve such that the composition is delivered to trigeminal ganglion and parts of the trigeminal brainstem nuclear complex, such as the subnucleus caudalis. In such intracellular pathways, the therapeutic agent may first be transported though nasal mucosa. Although antibody fragments that include the Fc portion (constant region) of an immunoglobulin may also be delivered by one of the aforementioned routes, one of the delivery routes may include being taken up by cells in the nasal mucosal epithelium having neonatal Fc receptors (FcRn) which may, depending on the mechanism, facilitate or hinder transport of the composition across the olfactory epithelium.

Extracellular pathways of entry of the composition into the central nervous system via the nasal cavity include direct entry into the cerebrospinal fluid, entry into the CNS parenchyma through channels, tracts or compartments associated with the olfactory system, such as the peripheral olfactory system, including the system that connects the nasal passages with the olfactory bulbs and rostral brain areas; and entry into the CNS parenchyma through channels, tracts or compartments associated with the trigeminal system, such as the peripheral trigeminal system, including the system connecting the nasal passages with the brainstem and spinal chord (Thorne, R. G. et al., Neuroscience 127:481-496 (2004)). Direct transport as used herein includes transport via one or more of the non-systemic pathways described herein.

Transport of the composition directly to the central nervous system by one or more of the mechanisms described herein allows the blood-brain barrier to be bypassed and overcomes the associated challenges and disadvantages surrounding systemic transport of agents to the central nervous system. Additionally, transporting the compositions by the methods described herein may allow less of the composition to be used as a greater proportion of the administered dose reaches the central nervous system target. In the case of administration of agents that are endogenously produced in the subject treated, the physiologic effects are typically comparable to the endogenous agent.

A therapeutically effective amount of the therapeutic composition is provided. As used herein, a therapeutically effective amount of the composition is the quantity of the composition required to achieve a specific therapeutic effect. For example, the amount is typically that required to reach a specified or desired clinical endpoint, such as a decrease in the progression of the disorder, a lessening of the severity of the symptoms of the disorder and/or elimination of the disorder. This amount will vary depending on the time of administration, the route of administration, the duration of treatment, the specific composition used and the health of the patient as known in the art. The skilled artisan will be able to determine the optimum dosage.

By intranasally administering the compositions by the methods described herein, it is realized that a smaller amount of the composition may be administered compared to systemic administration, including intravenous, oral, intramuscular, intraperitoneal, transdermal, etc. The amount of active agent and/or compositions required to achieve a desired clinical endpoint or therapeutic effect when intranasally administered as described herein may be less compared to systemic administration. Additionally, upon administering the compositions intranasally in the delivery and treatment methods described herein, about 5-fold to about 500-fold, and further about 10-fold to about 100-fold, less systemic exposure may be obtained compared to administration of the same amount systemically. Furthermore, at least about 5-fold, further at least about 10-fold, preferably at least about 20-fold and further at least about 50-fold less systemic exposure may be obtained compared to administration of the same amount systemically. In determining the therapeutic effectiveness of the compositions, clinical endpoints known to the art for the particular disorder may be monitored. For example, suitable clinical endpoints for Alzheimers' disease include, for example, decreases in memory loss, language deterioration, confusion, restlessness and mood swings; and improved ability to mentally manipulate visual information as determined by standard methods.

Suitable clinical endpoints for Huntington's disease include a decrease in uncontrolled movements, and an improvement or no further decrease of intellectual faculties.

Suitable clinical endpoints for Parkinson's disease include, for example, a decrease in the characteristic tremor (trembling or shaking) of a limb, especially when the body is at rest, an increase in movement (to help overcome bradykinesia), improved ability to move (to help overcome akinesia), less rigid limbs, improvement in a shuffling gait, and an improved posture (correcting the characteristic stooped posture). Such clinical endpoints may be observed by standard methods. Other suitable clinical endpoints include a decrease in nerve cell degeneration and/or no further decline in nerve cell degeneration and may be observed, for example, by brain imaging techniques, including computer assisted tomography (CAT) scanning, magnetic resonance imaging methods, or similar methods known to the art.

Suitable clinical endpoints for obesity include, for example, a decrease in body weight, body fat, food intake or a combination thereof.

Suitable clinical endpoints for sleep disorders, such as insomnia, include, for example, an improvement in the ability to sleep, and especially improved rapid eye movement (REM) sleep.

Suitable clinical endpoints for autoimmune disorders such as multiple sclerosis include, for example, a decrease in the number of brain lesions, increased extremity strength or a decreased in tremors or paralysis of extremities. Decreases in the number of brain lesions may be observed by brain imaging techniques previously described herein. Other suitable clinical endpoints include a decrease in inflammation of nervous tissue which may be determined by, for example, lumbar puncture techniques and subsequent analysis of cerebrospinal fluid known to the art.

In individuals who have experienced a stroke, a suitable clinical endpoint includes an increase in blood flow in the affected blood vessel as determined by computer tomographic methods as known in the art and as described, for example, in Nabavi, D. G., et al., Radiology 213:141-149 (1999). A further clinical endpoint includes a decrease in numbness in the face, arm or leg; or a decrease in the intensity of a headache associated with the stroke. Yet another clinical endpoint includes a decrease in the cell, tissue or organ damage or death due to the stroke. Such decrease in cell or tissue damage may be assessed by brain imaging techniques previously described herein, or similar methods known to the art.

Suitable clinical endpoints in neuropsychologic disorders such as schizophrenia include, for example, improvements in abnormal behavior, and a decrease in hallucinations and/or delusions.

The patient or subject treated according to the methods of the present invention is typically one in need of such treatment, including one that has a particular disorder amenable to treatment by such methods. The patient or subject is typically a mammal, such as a human, although other mammals may also be treated.

### EXAMPLES

Reference will now be made to specific illustrative examples. It is to be understood that the examples are provided to illustrate preferred embodiments and that no limitation to the scope is intended thereby. Additionally, all documents cited herein are indicative of the level of skill in the art and are hereby incorporated by reference in their entirety.

### Example 1

### Brain Distribution of α-Melanocyte Stimulating Hormone Mimetibody After Intranasal Administration

This example shows that an α-melanocyte stimulating hormone mimetibody (α-MSH mimetibody) is transported to various regions in the brain and was detected at about 25 minutes after intranasal administration while reducing systemic exposure according to the methods of the present invention. The example further shows that the α-MSH mimetibody delivered to the brain is retained in the brain for at least up to 5 hours post-delivery.

Methods

An α-MSH mimetibody was prepared, to serve as a model and exemplary therapeutic compound to illustrate the claimed method. The α-MSH mimetibody is a homo-dimeric fusion molecule that consists of the therapeutic α-MSH polypeptide, identified herein as SEQ ID NO:1, and the Fc portion of the human immunoglobulin G1 (IgG1) monoclonal antibody. The engineered fusion polypeptide was produced using recombinant DNA methods.

The α-MSH mimetibody was iodinated by Amersham Biosciences's Iodine-125 Custom Labeling Services using the Chloramine T method. ¹²⁵I-labeled α-MSH mimetibody, together with unlabeled α-MSH mimetibody as a cold carrier, was intranasally or intravenously administered to eight anesthetized rats (Sprague Dawley, 200-250 g). Intranasal drug administration was performed in the fume hood behind a lead-impregnated shield. Each rat was placed on its back on a heating pad with a 37° C. rectal probe; the rat's head was slightly elevated by rolled-up 4×4 gauze. The unlabeled mimetibody, dissolved in PBS, was spiked with 39 μCi of ¹²⁵-I labeled α-MSH mimetibody. A total volume of 100 μl containing approximately 13 nmol or 0.8 mg of α-MSH mimetibody was administrated in 10 μl nose drops to alternating nares every two minutes over a 15-20 minute time period to young male rats while under anesthesia and lying on their back. For intravenous administration, ¹²⁵I-labeled α-MSH mimetibody was delivered as a bolus injection through the tail vein in a total volume of 0.5 ml (diluted in saline). Rats were administered either a full dose (equivalent to intranasal) or 1/10^{th} of the intranasal dose (0.08 mg or 1.3 nmol α-MSH mimetibody containing 39 μCi). Blood samples were taken every 5 minutes up to 25 minutes. At about 27 minutes or 5 hours after the beginning of drug administration, the rats were perfused to remove blood-borne label and fixed.

The distribution of ¹²⁵I-labeled α-MSH mimetibody in the CNS and peripheral organs was assessed following intranasal or intravenous delivery in rats. Tissue pieces from the brain, organs and peripheral tissues were carefully excised, weighed and gamma-counted. Concentrations of α-MSH mimetibody were assessed using either gamma counting (quantitative analysis) or by autoradiography of coronal brain section (qualitative analysis). The nanomolar concentration in each tissue piece and in the blood was determined based on the amount of counts per tissue weight and specific activity of the radio-labeled protein.

Results

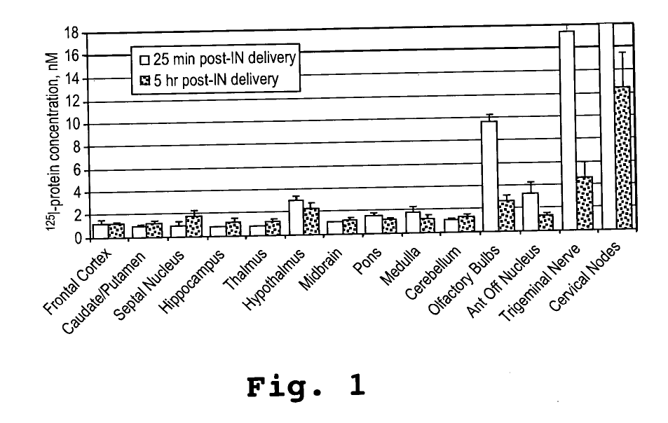

As seen in FIG. 1, the ¹²⁵I-labeled α-MSH mimetibody can be detected in various CNS tissues after intranasal delivery into young male rates within 25 minutes after administration. FIG. 1 further shows that most of the ¹²⁵I-labeled α-MSH mimetibody is retained at 5 hours post-intranasal delivery, suggesting that the half-life of α-MSH mimetibody is greater than 5 hours. It is more specifically seen that the ¹²⁵I-labeled α-MSH mimetibody reached the hypothalamus, the target site for action of the α-MSH peptide (binding to MCR4 on hypothalamic neurons). In addition, the hypothalamus (3 nM of mimetibody) is targeted with intranasal delivery although there is significant delivery to all brain regions, especially the medulla, pons and frontal cortex

Table 1 further compares the distribution of ¹²⁵I-labeled α-MSH mimetibody administered intranasally and intravenously.

TABLE 1
Distribution of α-MSH Mimetibody After Intranasal and Intravenous
Delivery

Average Concentration
of αMSH-Mimetibody (nM)

| | Intranasal | Intravenous |
|---|---|---|
| Tissue | (13 n mol) | (1.3 n mol) |
| blood sample 1 (5 min) | 0.5 +/− 0.1 | 33.4 +/− 2.97 |
| blood sample 2 (10 min) | 1.6 +/− 0.2 | 35.5 +/− 3.18 |
| blood sample 3 (15 min) | 2.9 +/− 0.4 | 32.1 +/− 2.83 |
| blood sample 4 (20 min) | 4.6 +/− 0.7 | 34.1 +/− 3.0.4 |
| blood sample 5 (25 min) | 5.4 +/− 0.8 | 28.2 +/− 2.59 |
| olfactory epithelium | 17.1 +/− 1.6 | 1.8 +/− 0.16 |
| olfactory bulb | 16.2 +/− 5 | 0.2 +/− 0.02 |
| trigeminal nerve | 19.1 +/− 3.4 | 0.5 +/− 0.03 |
| frontal cortex | 1.3 +/− 0.3 | 0.2 +/− 0.02 |
| Medulla | 1.8 +/− 0.4 | 0.1 +/− 0.01 |
| Hypothalamus | 3.0 ± 0.4 | 0.4 ± 0.06 |
| Liver | 1.8 +/− 1.0 | 18.9 +/− 1.59 |
| Kidney | 3.3 +/− 0.5 | 5.7 +/− 0.58 |
| Spleen | 1.2 +/− 0.2 | 3.9 +/− 0.76 |

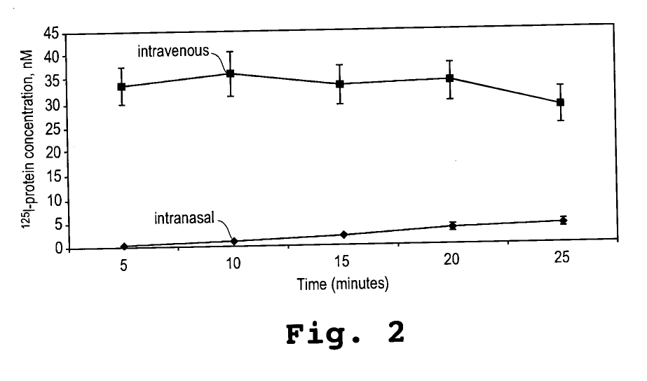

Intravenous delivery also targets the hypothalamus. However, despite the 13.5 higher blood exposure (AUC) with intravenous administration (see Table 1 and FIGS. 1 and 2), intranasal administration results in greater CNS delivery. Delivery of the peptide to the hypothalamus, frontal cortex, and medulla were 7.5, 6.5 and 18 fold higher, respectively, with intranasal than intravenous administration.

Table 2 shows the relative effectiveness of intranasal (i.n.) and intravenous (i.v.) delivery by comparing various ratios of polypeptide tissue concentrations. Specifically, the ratio of polypeptide concentration in the hypothalamus to polypeptide concentration in the blood at 25 minutes post delivery is shown in Table 2, for both intranasal and intravenous delivery. The ratio of polypeptide 15 concentration in the hypothalamus to polypeptide concentration in the liver at 25 minutes post delivery is also shown in Table 2, for both intranasal and intravenous delivery. Intranasal delivery was significantly more effective, as evidenced by the 48 and 75 fold ratios, to deliver the polypeptide to the hypothalamus than was intravenous delivery.

TABLE 2
Relative effectiveness of intranasal and intravenous delivery in
targeting the hypothalamus

| | | | Ratio |
|---|---|---|---|
| | i.n.* | i.v* | (i.n.)/(i.v.) |
| [polypeptide]_{hypothalamus}/[polypeptide]_{blood} | 0.558 | 0.012 | 48 |
| [polypeptide]_{hypothalamus}/[polypeptide]ₗᵢᵥₑᵣ | 1.640 | 0.022 | 75 |

*i.n. = intranasal; i.v. = intravenous

The data in Table 1 and FIG. 2 also show that systemic exposure of the ¹²⁵I-labeled α-MSH mimetibody was low when administered intransally. An intranasal one-tenth the amount of the intravenous dose resulted in a 13.5-fold lower systemic exposure, based on the blood AUC(intravenous)/AUC(intranasal) ratio, and a 10.5-fold lower exposure based on a ratio of liver protein concentration when dosed intravenously to the liver protein concentration when dosed intranasally. Further a consistent depot of the mimetibody (17.1 ± uM) was created in the olfactory epithelium across the 14 animals (see Table 1 above), and olfactory and trigeminal pathway concentrations of the test protein were similar upon intranasal administration indicating that the protein travels to the CNS via the olfactory and trigeminal neural pathways. Comparing equal intranasal and intravenous doses, systemic exposure was about 96-fold lower based on blood AUC(i.v.)/AUC(i.n.) ratio with approximately equal amounts of protein delivered to the CNS and hypothalamus.

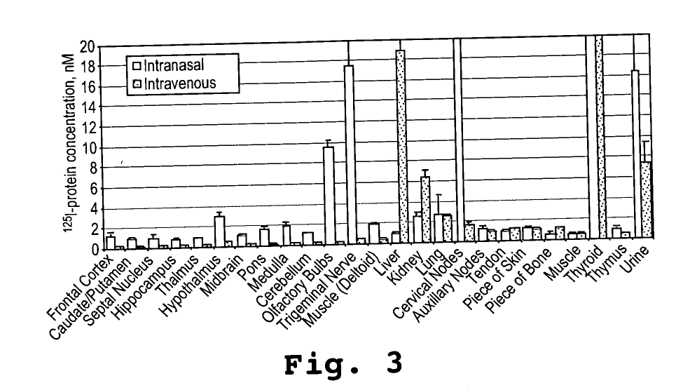

FIG. 3 shows that delivery of the ¹²⁵I-labeled α-MSH mimetibody to the central nervous system is unlikely to be secondary through the blood. For example, as seen in FIG. 3, when rats are exposed to a 10-fold higher dosage of ¹²⁵I-labeled α-MSH mimetibody by intranasal administration compared to intravenous administration, there was a higher accumulation of the ¹²⁵I-labeled α-MSH mimetibody in the central nervous system by intranasal administration.

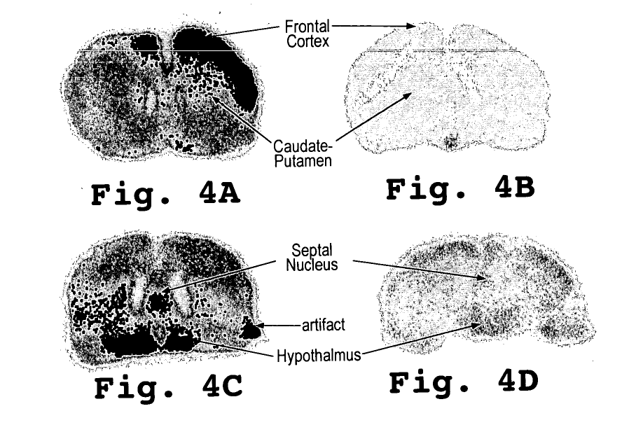

FIGS. 4A-4D show computer-generated autoradiographs of coronal sections of the rat brains 25 minutes after administration of ¹²⁵I-α-MSH mimetibody intranasally (FIGS. 4A, 4C) or intravenously (FIGS. 4B, 4D). The darkened area in the autoradiographs corresponds to the regions of high image intensity, which correlates to regions of fusion polypeptide delivery. As seen in FIGS. 4A, 4C, which correspond to the animals treated intranasally, the highest image intensities were observed in the olfactory tracts, hypothalamus, and frontal cortex. These images confirm findings from quantitative measurements.

### Example 2

### Dose-Dependent Reduction in Cumulative Food Intake in Normal Rats After Intranasal Administration of Alpha-MSH

This example shows that intranasal administration of a single dose of the N-acetylated α-melanocyte stimulating hormone (Ac-Ser-Tyr-Ser-Met-Glu-His-Phe-Arg-Trp-Gly-Lys-Pro-Val-NH2, SEQ ID NO:1, supplied by Phoenix Parmaceuticals, INC) was sufficient to achieve a dose dependent, pharmacodynamic response; specifically, a reduction of cumulative food intake, with an ED₅₀ at 24 hours of 6-7 nmol.

Methods

Two groups of nine rats each were assembled. In a cross-over design, each week one group was dosed with a phosphate buffered saline (PBS) vehicle and the other group was dosed with α-MSH peptide; the following week the treatment administered to each group was reversed. Prior to the study, the light cycle was slowly reversed, within a 2 weeks acclimation period. Rats were fasted for 24 hours prior to each experiment (water was always available), and received anesthesia 30 minutes prior to the beginning of the dark cycle (or the period of lights off). A single dose of drug ranging from 2.5 to 50 nmols or phosphate-saline buffered vehicle control was intranasally administered during anesthesia over approximately 20 minutes, similar to the procedure set forth in Example 1. Rats were placed on their backs on a heating pad and monitored until they become active, and then were placed in their cages with pre-weighed amounts of food. Food intake measurements were taken at 2, 4, 8, 24, 48 and 72 hours. Water intake and body weight were determined at 24 and 48 hours post-dosing.

Results and Conclusions

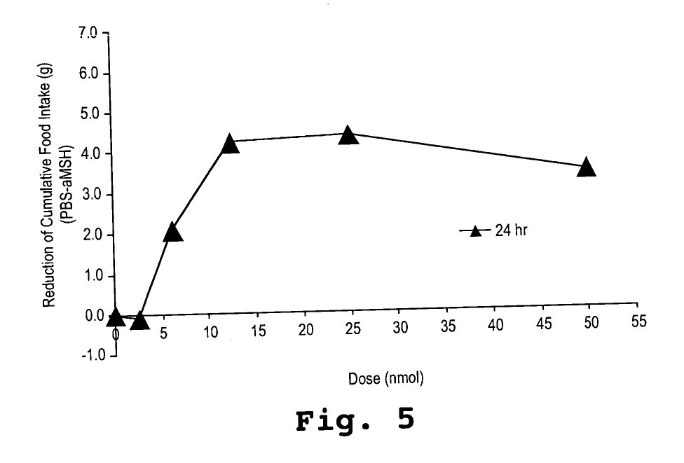

As seen in FIG. 5, intranasal α-MSH peptide reduces cumulative food intake dose dependently between 2.5-25 nmols at 24 hours with an ED₅₀ at 6-7 nmols.

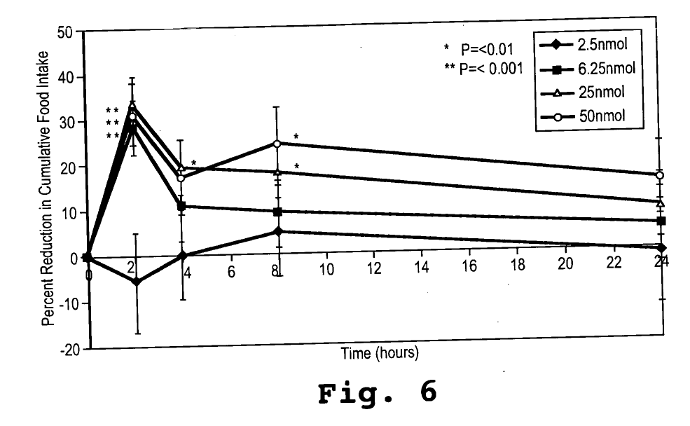

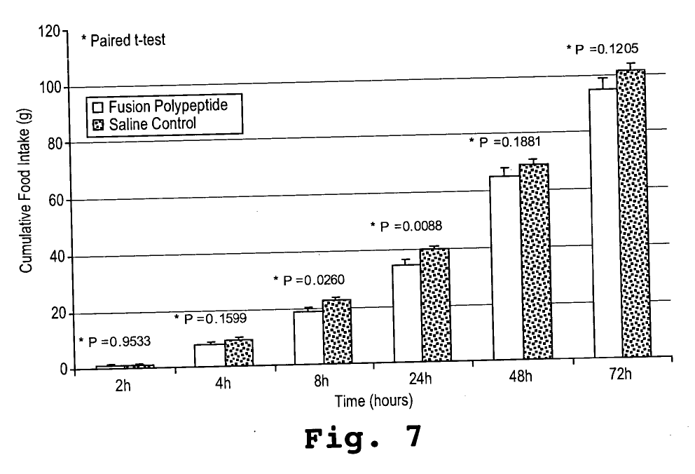

As shown in FIG. 6, a single dose of 25-50 nmol was maximally effective in reducing percent cumulative food intake. The 25 nmol dose reduced cumulative food consumption by 30% at 2 hours, by 18% at 8 hours, and by 9% at 24 hours. Water consumption and body weight remained unchanged. This study shows a dose dependent pharmacodynamic effect of a polypeptide after intranasal administration to a mammal.

### Example 3

### Reduction in Cumulative Food Intake in Normal Rats After Intranasal Administration of Alpha-MSH Mimetibody

This example shows that intranasal administration of a single dose of 25 nmols (5 mg/kg) of the α-MSH mimetibody is sufficient to reduce cumulative food intake significantly at 8 and 24 hours. Water consumption and body weight remained unchanged.

Methods

The study protocol and methods used were the same as described in Example 2. The total number of rats was 14.

Results and Conclusions

As seen in FIG. 7, a single dose of 25 nmol of intranasally delivered alpha-MSH mimetibody had a significant effect on decreasing cumulative food intake at 8 and 24 hours, with a non-statistically significant trend toward reduction at 48 and 72 hours. The significance at the later time points was likely lost due to the relatively small number of animals used in the study (n=14). The study shows that a 62 kDa large protein, like the α-MSH mimetibody, can be delivered to the CNS via the nasal route of administration.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

What is claimed is:

1. A method of delivering a therapeutic composition to the central nervous system of a mammal, comprising intranasally administering a therapeutically effective amount of a composition comprised of a therapeutic polypeptide and an antibody fragment.

2. The method of claim 1, wherein said composition is absorbed across nasal epithelium.

3. The method of claim 1, wherein said polypeptide is selected from a melanocortin receptor agonist, a growth hormone releasing factor receptor agonist, a vasopressin receptor agonist, a hormone peptide YY agonist, a neuropeptide Y receptor agonist, and an erythropoietin receptor agonist.

4. The method of claim 1, wherein said polypeptide is selected from melanocortin receptor antagonist, a growth hormone releasing factor receptor antagonist, a vasopressin receptor antagonist, a hormone peptide YY antagonist, a neuropeptide Y receptor antagonist, or an erythropoietin receptor antagonist.

5. The method of claim 3, wherein said melanocortin receptor agonist is melanocyte stimulating hormone peptide and said therapeutic composition is transported to the hypothalamus.

6. The method of claim 1, wherein said polypeptide is a melanocortin receptor antagonist and said therapeutic composition is transported to the hypothalamus.

7. The method of claim 1, wherein said antibody fragment is selected from the group consisting of an IgG fragment, IgE fragment, an IgM fragment, an IgA fragment, and an IgD fragment.

8. The method of claim 7, wherein said fragment comprises a constant region of an antibody selected from the group consisting of IgG, IgM, IgA, IgE, and IgD.

9. The method of claim 1, wherein said polypeptide is linked to said antibody fragment.

10. A method for targeting a polypeptide to the central nervous system, comprising
attaching an antibody or antibody fragment to the polypeptide to form a fusion polypeptide; and
administering the fusion polypeptide intranasally.

11. The method of claim 10, wherein the polypeptide is a therapeutic polypeptide.

12. The method of claim 10, wherein the antibody or antibody fragment is a therapeutic antibody or antibody fragment.

13. method of claim 10, wherein the polypeptide is hydrophobic and has a molecular weight of less than about 25 kDa.

14. A method of treatment, comprising intranasally administering to a mammal a therapeutically effective amount of a composition comprised of a polypeptide linked to an antibody fragment.

15. The method of claim 14, wherein said treatment is for a disorder that may be treated by administering a composition to the central nervous system of said mammal.

16. The method of claim 14, wherein said disorder is a metabolic or endocrine disorder.

17. The method of claim 16, wherein said metabolic or endocrine disorder is obesity or anorexia.

18. The method of claim 14, wherein said disorder is one that results in memory loss or loss in locomotion.

19. The method of claim 14, wherein said disorder is a neurodegenerative disorder.

20. The method of claim 19, wherein said neurodegenerative disorder is selected from Alzheimer's disease, Parkinson's disease, and Huntington's disease.

21. The method of claim 14, wherein said disorder is a sleep disorder or is due to acute brain injury.

22. The method of claim 21, wherein said sleep disorder is insomnia and said acute brain injury is from a stroke.

23. The method of claim 14, wherein said composition is absorbed into the nasal epithelial tissue.

24. The method of claim 14, wherein said intranasal administration achieves delivery of the composition to the central nervous system by an olfactory pathway or by a trigeminal neural pathway.

25. The method of claim 14, wherein said polypeptide is selected from a melanocortin receptor agonist, a growth hormone releasing factor receptor agonist, a vasopressin receptor agonist, a hormone peptide YY agonist, a neuropeptide Y receptor agonist, and an erythropoietin receptor agonist.

26. The method of claim 14, wherein said polypeptide is a melanocortin receptor agonist and the composition is transported to the hypothalamus.

27. The method of claim 14, wherein said antibody fragment is selected from the group consisting of an IgG fragment, an IgE fragment, an IgM fragment, an IgA fragment, and an IgD fragment.

28. The method of claim 27, wherein said fragment comprises a constant region from an antibody selected from the group consisting of IgG, IgE, IgM, IgA, and IgD.

29. A method of treatment, comprising intranasally administering to a mammal a therapeutic composition comprising a therapeutically effective amount of an antibody or an antibody fragment.


SEQUENCE LISTING (1 sequence)

SEQ ID NO: 1 - protein, 13 aa, Homo sapiens
SYSMEHFRWGKPV